(19) United States
(12) Patent Application Publication
(10) Pub. No.: US 2008/0287912 A1
(43) Pub. Date: Nov. 20, 2008

(54) Method, Device And Formulation For The Treatment Of Nail Disorders

(75) Inventor: David John Horrigan, Encinitas, CA (US)

Correspondence Address: HORRIGAN CORPORATION, 2300 EIGHTH ST., OLIVENHAIN, CA, 92024, US

(73) Assignee: Horrigan Corporation, Encinitas, CA (US)

(21) Appl. No.: 11/747,997

(22) Filed: May 14, 2007

(51) Int. Cl. A61M 31/00; A46B 9/00; A61B 17/00

(52) U.S. Cl. 604/514; 15/167.1; 604/48; 604/500; 606/1

(57) ABSTRACT

An improved method, device and formulation for the treatment of nail disorders characterized by de-lamination, thickening or crumbling of the nails. The invention consists of a tubular proboscis (12) attached to a reservoir (13) filled with fluid biocide or cleaning agent that can be inserted under the nail (15) and into the nail cavity (10) and that fluid is forcibly flushed (14) via the proboscis, under the nail, removing contamination (24) and applying a biocide to kill remaining infectious agents (24). The method also includes the use of a specifically designed brush (23) to aid in the removal of built-up carotin waste (11) that manifests as nail thickening and causes cavity formation. The advantages of the invention are that it allows the biocide access the infected area and allows cleaning of the cavity which facilitates re-growth of the nail to the bed.

### CROSS-REFERENCE TO RELATED APPLICATIONS PPA 60/746,799

### SEQUENCE LISTING OF PROGRAM

Not Applicable

### FIELD OF THE INVENTION

This invention relates to the treatment of nail disorders, specifically to such disorders that are difficult to treat due to inability to access the infected or damaged site.

### BACKGROUND OF THE INVENTION—PRIOR ART

Treatment for nail disorders prior to this invention consisted of the topical application of a biocide or the internal ingestion of a targeted antibiotic or fungicide. These methods have been marginally effective due to the fact that the fungi or contaminants causing most of these disorders exist in the space between the nail and the nail bed and are insulated from the medicine.

Historically, a common nail brush has also been used to scrub the outer surface of the infected nail but, again, this device lacks the design to effectively access the areas that need to be cleaned.

In addition to their relative ineffectiveness, the internally-ingested prescription treatments have occasionally proven to be hazardous to certain human organs such as the liver or heart.

Known fungal organisms causing onychomycosis-type nail disorders include Epidermophyton floccusum, Trichophyton rubrum, Trichophyton mentagrophytes, Trichophyton megninii, Trichophyton schoenleinii, Trichophyton tonsurans and Candida albicans. Although numerous systemic and topical medications have been disclosed previously for use in treating onychomycosis, most are ineffective, cause unwanted side effects, or are prohibitively expensive.

A number of electromagnetic and ultraviolet radiation treatments such as those described in U.S. Pat. No. 6,129,893 (Bolton, 2000) and U.S. Pat. No. 6,960,201 (Cumbie 2005) have been proposed and as much as they are effective at killing or rendering the fungus impotent, they fail to deal with the contained residue that prevent the nail from re-growing or reattaching to the nail bed.

In U.S. Pat. No. 6,846,837 Malbach, et al teaches that formulating a fungicide to permeate the nail effectively solves the problem as does Sorenson, et al (U.S. Pat. No. 5,840,283) and Sun, et al (U.S. Pat. No. 6,042,845) teaches that formulating for permeability and using a bandage to apply the biocide. But again, they fail to take into account the residue under the nail that prevents the nail from re-growing or the keratin buildup that contains spores or other infectious agents that can later emerge and re-infect the site.

Feldman in U.S. Pat. Nos. 6,572,580 and 6,264,628 grasps the necessity of accessing the nail bed and teaches using a device that notches the nail so that fungicide can penetrate but then uses a patch device to infuse the biocide. Karell (U.S. Pat. No. 5,947,956) also penetrates the nail using a laser device. These methods do not address the necessity of removing the contaminants nor does it allow for accessing very confined fungal cavities.

Berenstein (U.S. Pat. No. 5,795,314) understands the necessity of forcing the fungicide into the cavity however his complex device shows no method to get around the carotin barrier normally found at the front of the nail nor does it offer any technique accessing fungal residue in extremely deep or narrow cavities.

The particular problems associated with treating nail disorders have not been successfully addressed by the prior art biocides, devices or cleaning methods.

Therefore it would be desirable to provide a method, device or devices and appropriate formulations that overcame these obstacles in a novel fashion.

### DEFINITION OF TERMS

Biocide—any antiseptic, antibiotic, fungicide, germicide or anti-viral suitable for use with the methods, devices, and formulations described herein.

Cleaning Agent—a soap, solvent, acid or alkali suitable for use with the methods, devices, and formulations described herein.

Softening Agent—a soap, solvent or acid suitable for use with the methods, devices and formulations described herein.

Nail De-lamination—or onycholysis, the phenomena where the nail plate has become detached from the nail bed due to impact or disease such as pseudomonas, fungal onychomycosis, tinea unguis, onycatrophia, onychauxis, or methyl methacrylate damage.

Fungal cavity—a confined space under the nail plate that is characterized by nail delamination with fungal residue building up on the undersurface of the nail plate at or near the distal extremity of the nail bed. This fungal residue provides a protected space under the nail for infectious agents to thrive.

Flushing—forcefully streaming fluid for the purpose of removing detritus or dissolving built up contaminants.

Proboscis—a tubular nozzle of sufficient size to be comfortably inserted under the nail plate and into the fungal cavity to dispense biocides, cleaners and cosmetic solutions and to facilitate access to and flushing of the cavity.

### SUMMARY OF THE INVENTION

Accordingly, it is a principal object of the present invention to overcome the disadvantages associated with prior art methods, devices and formulations and provide a simple therapeutic nail treatment method, device and formulation to accomplish this. The present invention infects a biocide/cleaner via a specifically designed nozzle that irrigates the infected or delaminated nail cavity, killing and/or removing any contaminant from within that would inhibit nail re-growth.

It is yet another object of the present invention to reduce nail thickening resulting from carotin buildup as a result of fungal infection or contamination by fungal residue by utilizing the specifically designed nozzle, a specifically formulated solvent and a specifically designed scrubbing device.

It is another object of the present invention to beautify nails that have become discolored as a result of fungal infection or contamination by fungal residue or accumulated dirt buildup by using a specifically designed nozzle and a specifically formulated cleaning solution.

It is yet another object of the present invention to provide a method, device and formulation for cleansing nails for the prevention of nail deterioration utilizing a specifically designed nail brush and a specifically designed formulation of cleaning fluid.

It is a further object of the present invention to provide a vehicle consisting of a flexible or pressurized fluid container with a dispensing proboscis for applying a biocide deep under the nail to kill fungus and treat infections hidden deep within a nail cavity.

It is an additional object of the present invention to provide an additional apparatus for the maintenance of fresh, clean nails consisting of a motorized disposable brush head that dispenses cleaning or biocidal fluid.

It is still another object of the present invention to provide a method, device and formulation to make nails cosmetically attractive during the nail re-growth period utilizing a specifically designed nozzle and a naturally colored gel that fills the cavity that can contain medicine to continue to treat infections and dissolve buildups.

It is yet another object of the present invention to provide variations suitable for use by medical professionals, aestheticians and general consumers.

It is still an additional object of the present invention to provide the above apparatus in a form that is easy-to-use for its intended market, pain free, inexpensive to manufacture and consistently effective.

Other features and advantages of the present invention will become apparent from the following drawings and description.

### BRIEF DESCRIPTION OF THE DRAWINGS

For a better understanding of my treatment for nail disorders with regard to the embodiments thereof, reference is made to the accompanying drawings, in which like numerals designate corresponding elements or sections throughout, and in which:

FIG. 1 illustrates a cutaway nail, showing a simplified version of the device with its tubular proboscis being inserted into a nail cavity while arrows illustrate the flushing out of contaminated fluid, removing fungus, fungal residue and other contaminants.

FIG. 2 illustrates a sectional rendering of a toe and nail illustrating residue buildup (11) and fungal cavity (10)

FIG. 3 illustrates a sectional drawing of a toe, nail, and device with tubular proboscis inserted into the fungal cavity.

FIG. 4 illustrates a pressurized dispenser with a flexible hose attached to a handle with a tubular proboscis attached.

FIG. 5 illustrates a narrow brush with long, firm bristles.

FIG. 6 illustrates a powered brush with long firm bristles.

FIG. 7 illustrates a powered brush with long firm bristles and a detachable, disposable head filled with treatment fluid.

FIG. 8 illustrates a plastic moulded fluid container that has the proboscis formed as part of the container.

FIG. 9 illustrates an insertion handle with a custom tubular proboscis for directing the flushing fluid and scraping the undersurface of the nail.

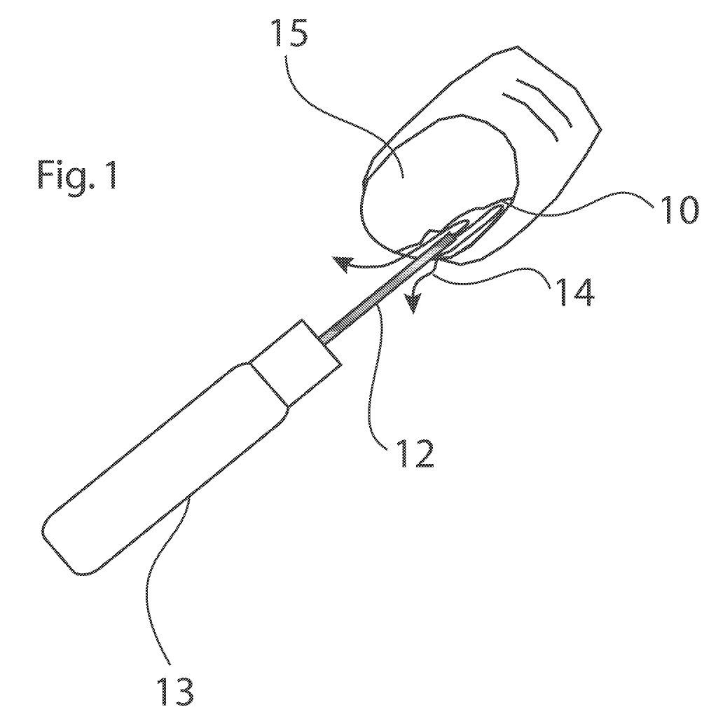

### DRAWINGS—LIST OF REFERENCE NUMERALS

FIG. 10 shows the fungal cavity, a space under the nail plate where the nail is de-laminated from the nail bed.

FIG. 11 shows a carotin buildup that protects and creates the fungal cavity.

FIG. 12 shows the tubular proboscis used for irrigating the fungal cavity.

FIG. 13 shows the container, flexible or pressurized, for the cleaner, biocide, solvent or gel.

FIG. 14 illustrates the flow of the cleaner, biocide or solvent as it removes contaminants.

FIG. 15 illustrates the nail plate.

FIG. 16 illustrates a manual brush handle.

FIG. 17 illustrates a powered brush handle.

FIG. 18 illustrates a removable/disposable powered brush head.

FIG. 19 illustrates tubular aspect of the brushes.

FIG. 20 rotating aspect of the brushes.

FIG. 21 illustrates the flexible supply line to flushing nozzle.

FIG. 22 illustrates the elongated aspect of the brush handle.

FIG. 23 illustrates the narrow aspect of the brush bristles.

FIG. 24 illustrates active fungus.

FIG. 25 illustrates the toe or finger.

FIG. 26 illustrates the medicinal/cleaner reservoir aspect of the brush head.

FIG. 27 illustrates the flow of the cleaner/medicine in use.

FIG. 28 illustrates a deflector port for redirecting the flow of cleaner/medicine/biocide.

FIG. 29 illustrates the tooth for scraping the underside of the nail.

FIG. 30 illustrates a ported plug for proboscis.

FIG. 31 illustrates the flow of the flushing fluid.

### DETAILED DESCRIPTION OF THE PREFERRED EMBODIMENT

As required, detailed embodiments of the present invention are disclosed herein, however, it is to be understood that the disclosed embodiments are merely exemplary of my treatment for nail disorders which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Reference is now made to the drawings, wherein like characteristics and features of the present invention shown in the various FIGURES are designated by the same reference numerals.

Referring to FIGS. (1), (3), (4), (8), and (9) illustrating the preferred embodiment for treating nail conditions characterized by partial nail de-lamination from the nail bed from a toe or finger (25) consisting of a container (13) with an essentially tubular proboscis (12) to be inserted into the fungal cavity (10) or de-lamination cavity and a fluid is then flushed into the cavity (10) wherein the fluid removes contaminants (14) and treats the cavity with a biocide. The fluid consists of a solvent for dissolving the residue, a biocide for killing any fungus or bacteria contained within and a bleaching agent to restore the natural color to the undersurface of the nail.

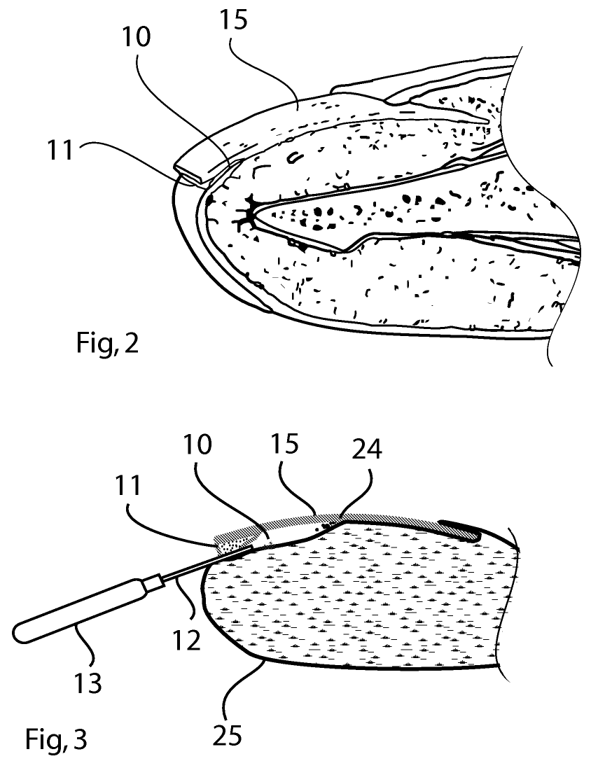

In FIG. 2, a sectional view of a toe illustrates the carotin buildup (11) under the nail plate (15) and the protected fungal cavity (10).

In FIG. 3, a carotin build up (11) on the undersurface of the nail plate (15) is illustrated and the proboscis is shown inserted under it into the cavity (10) so as to have biocidal and cleaning access to the active fungal colony (24).

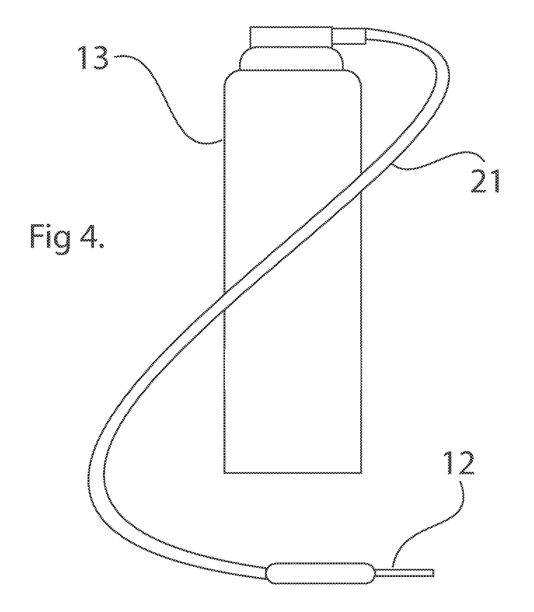

In FIG. 4 a larger fluid container is illustrated and is the ideal embodiment of my treatment for nail disorders for professionals treating the condition. This embodiment includes a flexible hose (21) and replaceable or sterilize-able proboscis (12) for treating multiple patients.

The preferred embodiment for treating nail conditions characterized by thickening of the nail includes the scrubbing of the under-fore-surface surface of the nail—the carotin buildup area—with a narrow brush (23). See also FIGS. (5), (6) and (7). In this case the injected fluid should be formulated to foam when brushed and aid in the dissolving of the buildup. In this embodiment the fluid should be thickened to remain on site after flushing and brushing.

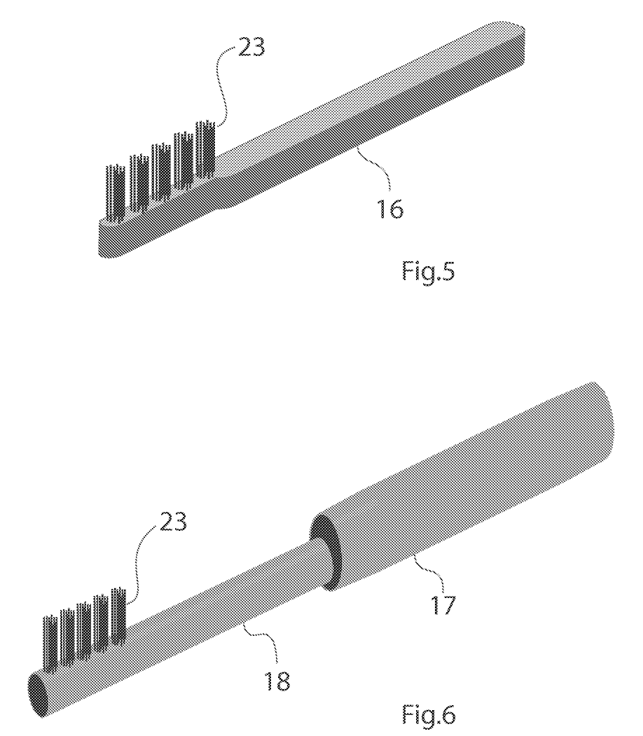

The brush should be as illustrated in FIG. 5, a brush that has preferably, a predominantly narrow bristle arrangement along the end of its handle (16), the bristles being less than one quarter of an inch wide, which is narrow as to compared to traditional dental-type cleaning brushes.

FIG. 9 illustrates a variation of the proboscis (28) that diverts the flushing fluid (31) via a ported plug (30) so as to offer better directional control toward both the fungal colonies and discoloration on the undersurface of the nail plate (9) and it also shows a raised edge (29) that can be used to scrape the nail while flushing to accomplish a more thorough cleaning in one application.

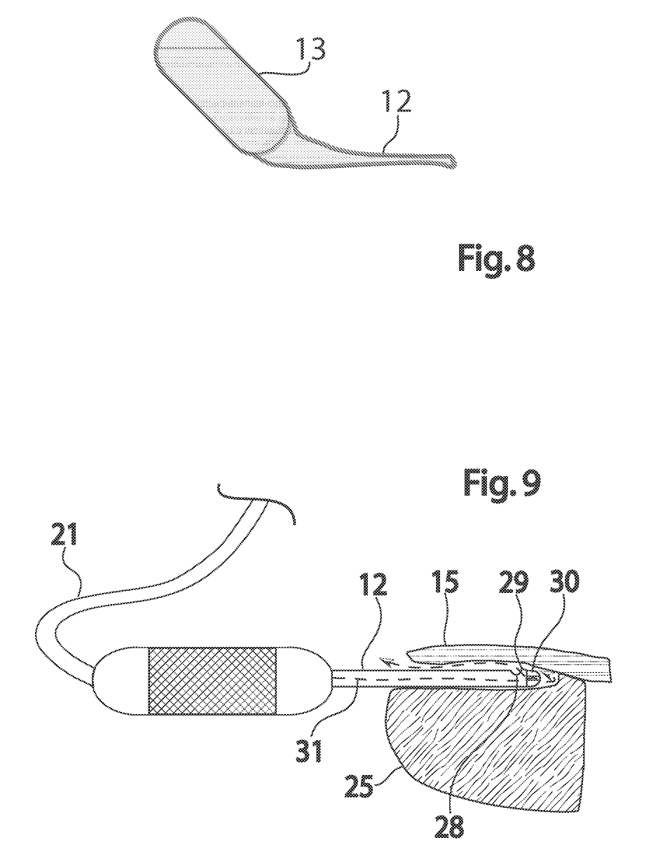

FIG. 8 illustrates a flexible, molded, plastic embodiment of the fluid container/proboscis that is the preferred embodiment of my treatment for nail disorders for individual use as it contains the reservoir (13) and the proboscis (12) in a single unit, with the proper dosage of the fluid for treatment in an inexpensive, simple to use, disposable form.

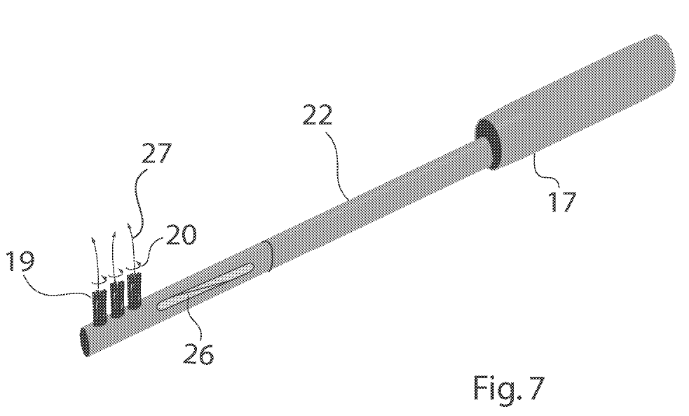

In situations where the nail infection is characterized by crumbling nails the same procedure and device is used as above, but the entire nail should be brushed frequently (see FIG. 5), and the biocide should be stronger or more targeted. In this case the preferred embodiment should be as above but a powered brush (FIGS. 6 and 7) should be used and this brush should have a powered handle (17) and an extended head (22) so the user, doesn't have difficulty reaching a long distance to comfortably brush nails that are awkward to reach such as toenails.

FIG. 7 illustrates the preferred embodiment of the powered brush with features such as a removable, disposable brush-head (18), and the elongated brush handle (22). Additionally, this embodiment will have a reservoir (26) for the medicine/cleaning fluid and tubular bristles (19) through which the fluid can be discharged directly (27) to the confined area of the carotin buildup and active fungal colony. It is preferred that the brush bristle bunches rotate or oscillate about a central axis (20) so as to target abrasion to the carotin build-up area. A back and forth linear motion of the bristles exerts non-useful force on the nail which may be subject to further de-lamination or becoming de-attached completely due to large fungal cavity spaces.

In the case of treating crumbling nails, the formulation could also be foamy to remove loose nail particles and leave a residue for continued biocidal action.

Cases where an individual has particularly cosmetically unappealing nails, the above treatment apply but with the additional step, after completion of the flushing, injecting or gel-like fluid into the cavity to fill the space and give the appearance that no cavity exists. The formulation in this case should be colored the same color as the nail bed or the nail plate and thickened so as to not run out for a few hours. The formulation can include a bleaching agent to improve the discoloration.

The preferred formula for the flushing fluid is a topically safe biocide, such as chlorhexidine gluconate, a 4% solution, or dimethyl benzyl ammonium chloride at a 0.13% solution and a bleaching agent such as 1-hydrogen peroxide at a 3-4% concentration and a solvent such as the surfactant sodium lauryl sulfate. The viscosity should be such that it flows freely into tight spaces. A viscosity of from 0.89 to 9 centipoise is preferred. Since the flushing or cleaning via an inserted proboscis is at new use for previously wiped-on, painted-on or ingested biocides and cleaners, this method of treatment could utilize and make them considerably more effective provided that they are formulated for use with this device.

The more formulation for the brushing fluid should be thicker and have more foaming effect. One example of how this could be accomplished is by increasing the surfactant in the mixture and adding a small amount of xanthan gum to the formulation increasing the viscosity to from 10 to 16 centipoise. Here a more targeted biocide would also be helpful such as terbinafine-HCl, naftifine-HCl, or Triconazole.

The preferred formulation for the cosmetic gel should have a viscosity of from 30 to 70 centipoise (at body temperature) and it need not have foaming agents added. The bleaching and dissolving characteristics should, however be retained. Since using a biocide injected as a cosmetic via an inserted proboscis is a new use for previously wiped-on or ingested biocides and cleaners this method of treatment can utilize them provided that they are formulated for use with this device.

### Removal of Non-Native Material

It was discovered during testing that spores from the parent infection became fused within the keratin waste that built up on the lower exterior surface of the nail causing the phenomena referred to as nail thickening. The spores were in an ideal environment to develop into a mature fungus given that they were encapsulated within their primary food source and also protected from biocides. It is believed that his is a significant reason why they condition was so difficult to treat. Months after you kill the parent infection, the spores emerge to re-infect the undernail cavity. Additional research discovered that using the citrus oil extract 4-Isopropenyl-1-methyl-1-cyclohexene (monocyclic terpene hydrocarbons) to bathe or soak under thickened nails effectively removed the non-native keratin buildup from the nail plate undersurface and as well as from the nail bed itself. With this keratin-based waste compound removed the parent fungal colony is exposed and more readily treated successfully.

### Professional Use

The preferred embodiment for the professional medical practitioner user could be a prescription biocide directed toward the specific virus, bacteria or fungus infecting the cavity or nail.

In all embodiments the proboscis should be disposable or sterilize-able to prevent others from becoming contaminated or infected. Similarly the preferred embodiment for the professional aesthetician would include single use or sterilize-able proboscis with the raised tooth (29) so that all discoloration could be removed in a single session.

The sectional shape of the proboscis need not be round. An oval shape would allow deeper insertion while maintaining good flushing flow rates. Other shapes would be suitable for better flushing characteristics or access.

### New Uses

This method for treating nail disorders constitutes a new use for dental style brushes with narrow rotating brushes as well as a new use for biocides that were previously applied externally or internally via ingestion such as terbinafine-HCl, naftifine-HCl, or Triconazole. The biocides would, of course, need to be formulated to be effective utilizing the methods as described and to be formulated to be used with the devices described herein.

### Theory

While I believe this to be an accurate theoretical explanation of the phenomena observed, I do not wish to be bound by this.

While developing the present invention it was discovered that certain nail fungi waste products, when mixed with water\produced an odiferous, slimy film that hardened into a tough nail-like plastic substance coating the under-surface of he nail causing it to thicken. This thickening, over time, would lift the nail from the bed creating a protected cavity in which the fungi could thrive, impervious to biocides applied externally or internally.

### Solution

To resolve this, the inventor discovered that by inserting a fine tube under the nail and into the cavity he could flush a biocide or cleaning solution under the nail and remove the fungus, its residue and any waste products contaminating the space.

### Re-Growth

Remarkably, with the cavity cleaned and fungus removed, the nail re-grew properly with the cavity disappearing over time as the nail re-grew. During case studies it was also noted that subjects that had nail de-lamination caused by impact and not disease also had their nails re-grow using this treatment.

### Cosmetics

Additionally any discoloration of the nail, apparently due to fungal deposits, also disappeared with continued treatment leaving the nail and bed a healthy pink color.

### Nail Deformation

The fungal residue, when mixed with water, builds up on the underside of the distal surface of the nail and forms a plastic thickening layer causing, when hardened and flexed, a lifting of the nail from the bed. In some cases the buildup can accumulate while not fully tearing the nail from the bed due to a particularly good bond. In this case the nail bed will deform and become extremely thickened so as to exceed one quarter of an inch in some cases. In this instance a softening agent is to be added to the flushing fluid to more quickly reduce the size of thickened nails and it was discovered that scrubbing with a narrow brush would facilitate the removal of built up keratin and debris, again improving the cosmetic appearance of the nail. This effect was enhanced when using a powered brush. As the residue is removed the nail returns to its normal shape.

### Harmless to Humans

The effectiveness of these methods, devices and formulations for the treatment of nail disorders greatly improves the efficacy of the treatment of such disorders and as a result, requires less biocide for a cure. Thus the formulations in their preferred embodiment should in all instances be harmless to humans.

### Interim Cosmetic Solution

It was also discovered that injecting a colorless gel into the cavity, the cavity would substantially disappear leaving a temporarily improved cosmetic appearance.

After initial treatment with flushing and after the nail resumed growing it was discovered that when a biocide was added to the gel re-growth was improved yet again.

### Veterinary Uses

These methods, devices and formulations for the treatment of nail disorders extensively address a long-standing human blight, but for animals, the loss or infection of a nail can be an even more devastating problem and thus I bring this technology to the attention of the veterinary community where it will hopefully alleviate suffering in animal species prone to inaccessible, cavity-type infections.

### Joy

As an unexpected side effect of brushing the toes and experiencing the revitalized cosmetic appearance of the nail using this treatment, testers reported experiencing an unusual joy and feeling of satisfaction lasting for many minutes after the treatment. Some users were virtually giddy with excitement at the results and with their renewed pride in their bodies.

### Scope of the Invention

Thus the reader will see that these methods, devices and formulations for the treatment of nail disorders within the invention provides an effective, reliable, inexpensive, simple to use, beautifying, relief from a persistent and stressful condition that has plagued mankind since his beginning.

While the above description contains many specificities, these should not be construed as limitations on the scope of my treatment for nail disorders, but rather as an exemplification of one preferred embodiment thereof. Many other variations are possible. For example the device could come in the form of a squirt gun with a mechanical pump and a retractable proboscis or simply a bottle with a hollow needle built into the cap. Perhaps a professional embodiment for treating hundreds of patients a day might include self-cleaning proboscis and a motorized fluid transfer and recovery/recycling system. It is also anticipate-able that a gel sheet could be inserted into the cavity by way of a proboscis to slowly dispense its self-contained biocide and flushing agent, eliminating the need for a fluid.

Accordingly the scope of my treatment for nail disorders should be determined, not by the embodiments illustrated, but by the appended claims and their legal equivalents.

I claim:

1. A method for treating nail de-lamination providing:
a. the insertion of a tube of predetermined size to fit under the nail and into the de-lamination cavity and
b. said tube having a means for attachment to a fluid reservoir and
c. a means for forcing such fluid from said reservoir through the tube into the cavity
whereby the cavity is substantially cleansed and foreign particles and micro-organisms are flushed out.

2. A method for treating nail diseases providing:
a. the insertion of a tube of predetermined size to fit under the nail and into the cavity and
b. said tube being affixed to a fluid reservoir filled with a biocide and
c. a means for forcing the biocide fluid into the cavity
whereby the cavity is substantially cleansed and foreign particles and micro-organisms are flushed out and micro-organisms are substantially killed.

3. A method for treating cosmetic nail abnormalities providing:
a. the insertion of a tube of predetermined size to fit under the nail and into the cavity and
b. said tube being affixed to a fluid reservoir filled with a viscous gel and
c. a means for forcing said viscous gel into the cavity
whereby the cavity is substantially filled with a gel eliminating the appearance of nail delamination.

4. A method for cleaning discolored nails providing:
a. the insertion of a tube of predetermined size to fit under the nail and into the discolored cavity and
b. said tube being affixed to a fluid reservoir filled with a cleaning fluid and
c. a means for forcing a cleaning fluid into the cavity
whereby the cavity is substantially cleansed of stains created by foreign particles and micro-organism residue.

5. A method for removing fungal residue and build-up under nails providing:
a. softening the residue with the application of a softening fluid and
b. scrubbing the underside of the nail with a brush
whereby the nail is substantially cleansed of residual build-up created by foreign particles and micro-organisms.

6. A device for treating nail diseases comprising
a. a tube of predetermined size to fit under a nail and into nail cavities created by disease or miss-treatment attached to
b. a container suitable for holding treatment fluid, and
c. a means for pressurizing said fluid for transfer through the tube and into the nail cavity
whereby said fluid can be flushed into the cavity substantially removing foreign particles and micro-organisms.

7. A device for cleaning discolored nails providing:
a. a tube of predetermined size to be inserted under the nail and into the discolored cavity and
b. said tube being affixed to a fluid reservoir filled with a fluid cleaning agent and
c. a means for forcing a cleaning agent into the cavity
whereby the cavity is substantially cleansed of stains created by foreign particles and micro-organism residue.

8. A device as in claim 7 but with the tube plugged and ported so as to direct the flushing flow to better evacuate detritus
whereby making the nail more cosmetically appealing with less effort.

9. A device as in claim 8 but with a scraping surface to assist in removal of discoloring buildup on the nail
whereby making the nail more attractive in one treatment.

10. A device as in claim 7 but with the tube substantially removable for sterilization or disposal
whereby eliminating cross-contamination of multiple users.

11. A predetermined formulation for treating nail diseases comprising a nonhazardous biocide made of a viscosity substantially low enough to flush foreign particles and micro-organisms from the cavity while substantially killing any that remain
whereby treating any infection within the nail cavity.

12. A predetermined formulation for treating nail diseases comprising a nonhazardous foaming biocide made of a viscosity substantially low enough to flush foreign particles and micro-organisms from the cavity while substantially killing any that remain while leaving a residue for continued effect
whereby continuing to treat any infection within the nail cavity.

13. A predetermined formulation for treating nail diseases comprising a nonhazardous cleaning agent made of a viscosity substantially low enough to flush foreign particles and micro-organisms from the cavity
whereby facilitating the re-growth of the nail plate to the nail bed.

14. A predetermined formulation for treating nail diseases comprising a nonhazardous foaming cleaning agent made of a viscosity substantially low enough to flush foreign particles and micro-organisms from the cavity while leaving a residue for continued effect
whereby facilitating continued re-growth of the nail plate to the nail bed.

15. A predetermined formulation for treating cosmetic damage resulting from nail diseases comprising a transparent gel that can be injected under the nail substantially filling the cavity
whereby giving the appearance that the nail-bed is not separated from the nail.

16. A device for treating nail diseases comprising
a. a collection of bristles narrowly assembled on
b. a handle
whereby allowing one to scrub in a confined space under a nail overhang or into cavities to substantially remove carotin buildup, partially dislodged foreign particles and externalized micro-organisms.

17. A device as in claim 16 but motor driven
whereby the use exerts less time and energy in the process.

18. A device as in claim 16 but with detachable, replaceable, brush heads
whereby the user can share a brushing device without cross-contamination.

19. A device as in claim 17 but with extended heads
whereby eliminating having to reach for those who have difficulty touching toes.

20. A device as in claim 18 but with a treatment fluid dispenser built into heads
whereby simplifying the procedure.

21. A device as in claim 20 but with bristles of a tubular nature
whereby allowing the dispensing of fluid directly to the surface to be treated or cleaned.

22. A device as in claim 16 but with an irrigation device as in claim 6 built in
whereby simplifying the procedure.

23. A device as in claim 17 but where the irrigation device built in and motor driven
whereby cleaning effort and time is reduced.

24. A predetermined formulation for treating nail diseases by the removal of non-native keratin buildup comprising either dilute or straight 4-Isopropenyl-1-methyl-1-cyclohexene (Monocyclic terpene hydrocarbons) applied directly to the buildup
whereby facilitating the biocidal access to the parent infection and the prevention of encapsulated spores or infectious agents from emerging to re-infect the nail.
